(19) United States
(12) Patent Application Publication
(10) Pub. No.: US 2015/0014150 A1
(43) Pub. Date: Jan. 15, 2015

(54) Process for Energy Recovery in Purifying Carboxylic Anhydride for Manufacturing Cellulose Esters

(71) Applicant: Celanese Acetate LLC, Irving, TX (US)

(72) Inventor: Denis G. Fallon, Blacksburg, VA (US)

(21) Appl. No.: 13/939,215

(22) Filed: Jul. 11, 2013

(51) Int. Cl. C07C 51/573; B01D 3/00

(52) U.S. Cl. CPC C07C 51/573; B01D 3/007
USPC 203/27

(57) ABSTRACT

Integration of a carboxylic anhydride purification system in the manufacturing of cellulose esters may include processes that includes distilling the crude carboxylic anhydride stream (that includes a carboxylic anhydride and a carboxylic acid) in a distillation column having an overhead or side stream comprising purified, vaporous carboxylic anhydride and a bottoms stream; heating a steam condensate stream in a steam generator to yield a low-temperature steam; and cooling at least a portion of the overhead or side stream to yield a cooled overhead or side stream. In some instances, a heat exchanger may be utilized in parallel or series with the steam generator.

### BACKGROUND

The present invention relates to integration of a carboxylic anhydride purification system in the manufacturing of cellulose esters with utility operations associated with the manufacturing unit.

The production of cellulose esters is derived from cellulose, generally wood pulp cellulose. For example in cellulose acetate, the cellulose is treated to provide maximum access to the cellulose structure and then reacted with acetic acid and acetic anhydride in the presence of a catalyst such as sulfuric acid. Other carboxylic anhydrides, such as butyric or propionic anhydride, may be utilized in combination with or alternative of acetic anhydride to react with the cellulose. Next, the reacted cellulose experiences partial hydrolysis to remove the sulfate and a sufficient number of carboxylic acid groups to give the product the desired properties. The anhydroglucose unit is the fundamental repeating structure of cellulose and has three hydroxyl groups that can react to form acetate esters. Where the final product has an acetate group on approximately two of every three hydroxyls, the product is known as diacetate. Where the final product has an acetate group on all three hydroxyls, the product is known as triacetate.

As part of the production process, relatively large quantities of both the carboxylic acid and the carboxylic anhydride are used. For this reason, many producers choose to purify the crude carboxylic anhydride as part of the unit operations for use in the acetylation procedure. Crude carboxylic anhydride must be cleaned of contaminates particularly heavy contaminates before use. These impurities, that may be present in trace amount, affect the quality of the carboxylic anhydride, which can cause the impurities to build up over time as the carboxylic anhydride is circulated through the reaction process.

Conventional purification techniques subject the crude carboxylic anhydride to column distillation. In many chemical processes such as carboxylic anhydride purification, distillation columns consume a significant amount of energy. The distillation columns may each independently receive the energy necessary to drive the separation within the column. The process of purifying the carboxylic anhydride uses a substantial amount of energy in order to separate the carboxylic anhydride from unwanted contaminates.

Accordingly, in view of the above considerations, there is a need to reduce the amount of energy needed to run the process or to somehow capture and reuse the energy that is put into the system. Any solution to the need must not negatively affect the carboxylic anhydride purification process itself or any associated units in the production facility.

### SUMMARY OF THE INVENTION

The present invention relates to integration of a carboxylic anhydride purification system in the manufacturing of cellulose esters with utility operations associated with the manufacturing unit.

One embodiment described herein is a process for the recovery of the heat from a carboxylic anhydride distillation column, the process including the steps of: providing a crude carboxylic anhydride stream comprising a carboxylic anhydride and a carboxylic acid; distilling the crude carboxylic anhydride stream in a distillation column having an overhead or side stream comprising purified, vaporous carboxylic anhydride and a bottoms stream; providing a steam generator comprising a first process inlet, a first process outlet, a first water inlet, and a first steam outlet; introducing at least a portion of the overhead or side stream to the steam generator via the first process inlet; introducing a steam condensate stream to the steam generator via the first water inlet; heating the steam condensate stream in the steam generator to yield a low-temperature steam; cooling the portion of the overhead or side stream to yield a cooled overhead or side stream; wherein the low-temperature steam exits the steam generator via the first steam outlet and the cooled overhead or side stream exits the steam generator via first process outlet; and wherein the temperature of the first process outlet is lower than the temperature of the first process inlet.

Another embodiment described herein is a process for the recovery of the heat from a carboxylic anhydride distillation column, the process including the steps of: providing a crude carboxylic anhydride stream comprising a carboxylic anhydride and a carboxylic acid; distilling the crude carboxylic anhydride stream in a distillation column having an overhead or side stream comprising purified, vaporous carboxylic anhydride and a bottoms stream; providing a steam generator comprising a first process inlet, a first process outlet, a first water inlet, and a first steam outlet; introducing at least a portion of the overhead or side stream to the steam generator via the first process inlet; introducing a steam condensate stream to the steam generator via the water inlet; heating the steam condensate stream in the steam generator to yield a low-temperature steam; cooling the portion of the overhead or side stream at least about 0.1° C. and up to about 190° C. to yield a cooled overhead or side stream; wherein the low-temperature steam exits the steam generator via the first steam outlet and the cooled overhead or side stream exits the steam generator via first process outlet; providing a heat exchanger comprising a second process inlet, a second process outlet, a second water inlet, and a second water outlet; introducing a second portion of the overhead or side stream to the second heat exchanger via the second process inlet; introducing a cooling water stream to the second heat exchanger via second water inlet; cooling the second portion of the overhead or side stream at least about 0.1° C. and up to about 190° C. in the second heat exchanger to yield a cooled second portion of the overhead or side stream; and wherein the cooled second portion of the overhead or side stream exits the second exchanger second process outlet and the cooling water stream exits the second heat exchanger via the second water outlet.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates an exemplary scheme according to one embodiment of the present invention.

FIG. 2 illustrates an exemplary scheme according to one embodiment of the present invention.

### DETAILED DESCRIPTION

In response to the need to recapture and reuse energy that is put into a carboxylic anhydride purification system, the present invention provides new and improved processes to advantageously increase the overall efficiency of the carboxylic anhydride purification process. The present invention captures the energy contained in a distillation overhead to produce low-pressure steam than can be used elsewhere in the cellulose esters production process. This invention integrates the energy needs of the carboxylic anhydride purification system in the manufacturing of cellulose esters with utility operations associated with the manufacturing process. As described above, anhydrides can include acetic anhydride, butyric anhydride, propionic anhydride, and the like, and combinations thereof for mixed anhydride processes (e.g., in producing a cellulose acetate-propionate).

Some embodiments of the present invention may involve transferring heat, preferably excess heat, from the carboxylic anhydride purification distillation column overhead, side, or other suitably hot stream exiting the distillation column to heat water (preferably steam liquid condensate) into low-pressure steam. In conventional systems, these streams that exit the distillation column would be cooled using cooling water and the excess heat would not be advantageously recovered. This recovery process will add to the efficiency of the overall carboxylic anhydride purification unit and the supporting utilities unit; thus decreasing the costs of fuel and energy consumption.

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, that will vary from one implementation to another, and would be a routine undertaking for those of ordinary skill in the art having the benefit of this disclosure.

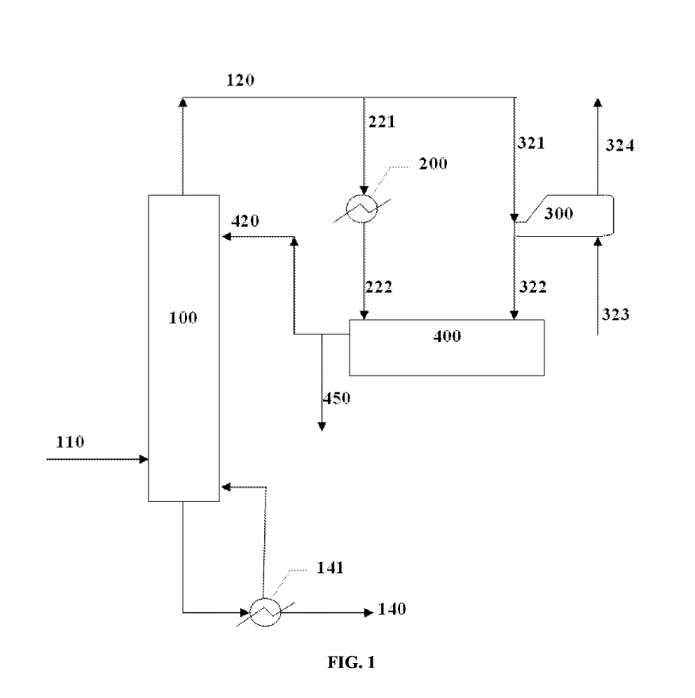

FIG. 1 shows an exemplary carboxylic anhydride purification unit. The principal unit operation is the distillation of crude carboxylic anhydride to remove light and heavy impurities. The carboxylic anhydride purification distillation column 100, receives a feed of crude carboxylic anhydride 110 comprising 40-99% carboxylic anhydride, the remainder being the carboxylic acid, trace higher boiling organic compounds, carbonaceous solids, and catalyst salts formed from the manufacture of carboxylic anhydride (typically 0.1-0.5%). Carboxylic anhydride purification distillation column 100 is typically a tray-style column. Column 100 includes a reboiler 141 at the bottom of the column that adds heat in order to drive a distillate overhead while allowing for the removal of heavy impurities from column 100 via heavy ends removal stream 140 containing carboxylic anhydride, carboxylic acid, concentrated carbonaceous solids, and catalyst salts. The overhead stream 120 comprises purified carboxylic anhydride with some carboxylic acid in a vapor form. As used herein, the term “overhead stream 120” may include not just the overhead itself, but also any side draw-off streams or other suitably hot stream exiting the distillation column that require cooling. For example one alternate configuration may include a vaporous side stream product containing purified carboxylic anhydride, the overhead containing mainly carboxylic acid, and the base product containing anhydride and heavy ends (e.g., solids). Both the side stream vapor and the overhead vapor are suitably hot enough to effectively generate low-pressure steam. However, anhydride light ends purification columns that principally remove compounds boiling at a lower temperature than carboxylic acids, generally do not exhibit a suitably high temperature generating low pressure steam.

It is desirable to cool and condense overhead stream 120 before it is sent for further processing. While this could be done using plant cooling water, the process of the present invention instead uses steam generator 300, through which at least a portion of overhead stream 120 is cooled using water from a utilities steam condensate via line 323. Thus, at least a portion of overhead stream 120 is condensed for further processing while simultaneously steam condensate experiences a desirable increase in temperature and pressure. Low pressure boiler feed make up water is sent to boiler steam generator 300 via line 323 and exits boiler steam generator 300 via line 324 as low pressure steam. On the process side of boiler steam generator 300, the cooled overhead stream exits the boiler steam generator 300 via line 322.

Depending on the volume of steam condensate available, all or a portion may be condensed via steam generator 300. As illustrated in FIG. 1, overhead stream 120 is split into a stream that is sent to the boiler steam generator 300 via line 321 and a stream that is sent to a cooling water heat exchanger 200 via line 221. One of skill in the art will recognize that where there is sufficient steam condensate to adequately cool overhead stream 120 via steam generator 300, then line 221 and cooling water heat exchanger 200 may not be necessary. Further, one of skill in the art will recognize the design implementations to allow for real-time changing of the amount of overhead stream 120 flow to each of boiler steam generator 300 and cooling water heat exchanger 200 (including flowing only through boiler steam generator 300).

The cooled overhead stream exits the cooling water heat exchanger 200 via line 222 and exits steam generator 300 via line 322, which are both sent to receiver 400. The liquid in the receiver is then either returned to distillation column 100 via line 420 or extracted from the unit operation in line 450.

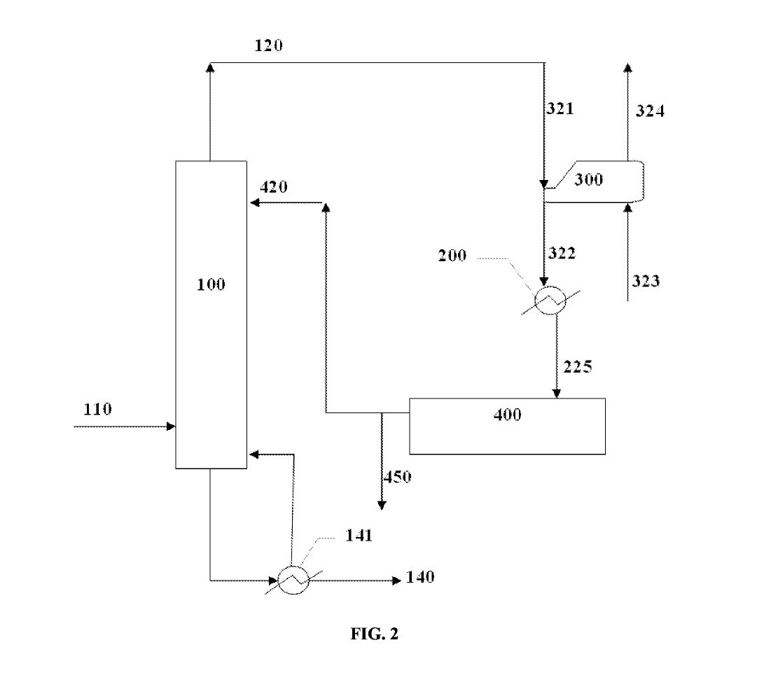

In an alternative embodiment shown in FIG. 2 with continued reference to FIG. 1, the boiler steam generator 300 and cooling water heat exchanger 200 operate in series rather than in parallel with the overhead stream 120 going first to boiler steam generator 300 through line 321 and then exiting boiler steam generator 300 through line 322 and going directly to cooling water heat exchanger 200. The cooled overhead stream then exits cooling water heat exchanger 200 via line 225 and is sent to receiver 400. This series embodiment may advantageously allow for additional heat recovery because a higher process temperature may be available at boiler steam generator 300.

In some embodiments, a hybrid of the foregoing embodiments illustrated in FIGS. 1-2 may be utilized where at least one cooling water heat exchanger is parallel with the boiler steam generator and at least one cooling water heat exchanger is in series with the boiler steam generator.

As noted, preferably 100% of overhead stream 120 is cooled using steam generator 300. However, the practical upper limit will depend on the operation of the particular unit at a particular site and the number and sizes of available distillation towers. Another practical consideration is that the operation should have an available cooling water heat exchanger to ensure cooling where boiler feed water make up flow can be variable. Having some fraction of cooling done by the more predictable cooling water flow can ensure better control of column cooling. In some instances, up to about 95% of overhead stream 120 may be cooled using boiler feed water heat exchanger 300 with at least 5% being cooled by a cooling water heat exchanger, which may enhance temperature stability in downstream processing (e.g., in the decanter).

Generally, overhead stream 120 ranges in temperature from about 50° C. to about 220° C. This range depends on the pressure and composition for the top vapor. Depending on the season and the location of the facility, steam condensate may range from about 30° C. to about 190° C. The quality and quantity of steam that can be produced in steam generator 300 depends upon a number of factors, including: the temperature of overhead stream 120, the temperature of the steam, and the respective volumes of steam condensate through steam generator 300 and the volume in line 321 sent through steam generator 300, and the exchanger design to maximize counter current heat transfer. In preferred embodiments, the steam condensate is converted into low-pressure steam having a pressure of at least about 1.5 psia to as high as about 165 psia. Atmospheric or higher-pressure steam can be used directly in the process for heating. Or it can be thermo-compressed using a higher steam pressure stream resulting in an intermediate pressure stream that may be more useful. Sub-atmospheric steam can be thermo-compressed in the same manner and utilized as such. The overhead stream 120 is preferably completely condensed in steam generator 300 and/or heat exchanger 200 and experiences a decrease in temperature of at least about 0.1° C. and up to about 190° C.

In some embodiments, the steam produced from generator 300 (optionally pressurized as described herein) may be utilized in heating carboxylic anhydride purification distillation column 100, using a mixture of the generated steam and other, high-pressure steam through the use of a thermocompressor. In some instances, the steam or a portion thereof may be routed to a steam line within the facility that may be routed back to this process and/or to other processes within the facility.

In some embodiments, the anhydride purification may be separated into two anhydride purification towers run in series rather than performed in a single purification column as illustrated in FIGS. 1-2. Where multiple purification towers are used, the heat recovery for the overhead stream performed in a steam generating heat exchanger (such as steam generator 300), either alone or in combination with a cooling water heat exchanger (such as cooling water heat exchanger 200), may be placed at the first column, the second column, or both.

One embodiment described herein is a process for the recovery of the heat from a carboxylic anhydride distillation column, the process including the steps of: providing a crude carboxylic anhydride stream comprising a carboxylic anhydride and a carboxylic acid; distilling the crude carboxylic anhydride stream in a distillation column having an overhead or side stream comprising purified, vaporous carboxylic anhydride and a bottoms stream; providing a steam generator comprising a first process inlet, a first process outlet, a first water inlet, and a first steam outlet; introducing at least a portion of the overhead or side stream to the steam generator via the first process inlet; introducing a steam condensate stream to the steam generator via the first water inlet; heating the steam condensate stream in the steam generator to yield a low-temperature steam; cooling the portion of the overhead or side stream to yield a cooled overhead or side stream; wherein the low-temperature steam exits the steam generator via the first steam outlet and the cooled overhead or side stream exits the steam generator via first process outlet; and wherein the temperature of the first process outlet is lower than the temperature of the first process inlet.

Optionally the foregoing embodiment may include at least one of the following elements in any combination: Element 1: the first process inlet having a temperature of between 50° C. and 220° C. and the first process outlet having a temperature of between 220° C. and 30° C.; Element 2: the first water inlet having a temperature of between 30° C. and 190° C. and the first water outlet having a temperature of between 30° C. and 190° C.; Element 3: the process further including providing a heat exchanger comprising a second process inlet, a second process outlet, a second water inlet, and a second water outlet; sending a second portion of the overhead or side stream to the second heat exchanger via the second process inlet; sending a cooling water stream to the second heat exchanger via second water inlet; cooling the second portion of the overhead or side stream in the second heat exchanger to yield a cooled second portion of the overhead or side stream, such that the cooled second portion of the overhead or side stream exits the second exchanger second process outlet and the cooling water stream exits the second heat exchanger via the second water outlet; wherein the temperature of the second process outlet is lower than the temperature of the second process inlet; and wherein the temperature of the second water outlet is higher than the temperature of the second water inlet; Element 4: Element 3 with the steam generator and the heat exchanger are in series with the first process outlet from the steam generator being sent to the second process inlet of the heat exchanger, and wherein the second portion of the overhead or side stream is the cooled overhead or side stream; Element 5: Element 3 with the steam generator and the heat exchanger are in parallel. By way of nonlimiting example, suitable combinations of elements may include, but are not limited to, Element 1 in combination with Element 3, Element 2 in combination with Element 3, Element 1 and 2 in combination, at least one of Elements 1 and 2 in combination with Element 4, at least one of Elements 1 and 2 in combination with Element 5, and so on.

Another embodiment described herein is a process for the recovery of the heat from a carboxylic anhydride distillation column, the process including the steps of: providing a crude carboxylic anhydride stream comprising a carboxylic anhydride and a carboxylic acid; distilling the crude carboxylic anhydride stream in a distillation column having an overhead or side stream comprising purified, vaporous carboxylic anhydride and a bottoms stream; providing a steam generator comprising a first process inlet, a first process outlet, a first water inlet, and a first steam outlet; introducing at least a portion of the overhead or side stream to the steam generator via the first process inlet; introducing a steam condensate stream to the steam generator via the water inlet; heating the steam condensate stream in the steam generator to yield a low-temperature steam; cooling the portion of the overhead or side stream at least about 0.1° C. and up to about 190° C. to yield a cooled overhead or side stream; wherein the low-temperature steam exits the steam generator via the first steam outlet and the cooled overhead or side stream exits the steam generator via first process outlet; providing a heat exchanger comprising a second process inlet, a second process outlet, a second water inlet, and a second water outlet; introducing a second portion of the overhead or side stream to the second heat exchanger via the second process inlet; introducing a cooling water stream to the second heat exchanger via second water inlet; cooling the second portion of the overhead or side stream at least about 0.1° C. and up to about 190° C. in the second heat exchanger to yield a cooled second portion of the overhead or side stream; and wherein the cooled second portion of the overhead or side stream exits the second exchanger second process outlet and the cooling water stream exits the second heat exchanger via the second water outlet.

Optionally the foregoing embodiment may include at least one of the following elements in any combination: Element 1: the first process inlet having a temperature of between 50° C. and 220° C. and the first process outlet having a temperature of between 220° C. and 30° C.; Element 2: the first water inlet having a temperature of between 30° C. and 190° C. and the first water outlet having a temperature of between 30° C. and 190° C.; Element 3: the steam generator and the heat exchanger being in series with the first process outlet from the steam generator being sent to the second process inlet of the heat exchanger, and wherein the second portion of the overhead or side stream is the cooled overhead or side stream; and Element 4: the steam generator and the heat exchanger being in parallel. By way of nonlimiting example, suitable combinations of elements may include, but are not limited to, Elements 1 and 2 in combination, at least one of Elements 1 and 2 in combination with Element 3, at least one of Elements 1 and 2 in combination with Element 4, and so on.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A process for the recovery of the heat from a carboxylic anhydride distillation column, comprising the steps of:
providing a crude carboxylic anhydride stream comprising a carboxylic anhydride and a carboxylic acid;
distilling the crude carboxylic anhydride stream in a distillation column having an overhead or side stream comprising purified, vaporous carboxylic anhydride and a bottoms stream;
providing a steam generator comprising a first process inlet, a first process outlet, a first water inlet, and a first steam outlet;
introducing at least a portion of the overhead or side stream to the steam generator via the first process inlet;
introducing a steam condensate stream to the steam generator via the first water inlet;
heating the steam condensate stream in the steam generator to yield a low-temperature steam;
cooling the portion of the overhead or side stream to yield a cooled overhead or side stream;
wherein the low-temperature steam exits the steam generator via the first steam outlet and the cooled overhead or side stream exits the steam generator via first process outlet; and
wherein the temperature of the first process outlet is lower than the temperature of the first process inlet.

2. The process of claim 1 wherein the first process inlet has a temperature of between 50° C. and 220° C. and the first process outlet has a temperature of between 220° C. and 30° C.

3. The process of claim 1 wherein the first water inlet has a temperature of between 30° C. and 190° C. and the first water outlet has a temperature of between 30° C. and 190° C.

4. The process of claim 1 further comprising:
providing a heat exchanger comprising a second process inlet, a second process outlet, a second water inlet, and a second water outlet;
sending a second portion of the overhead or side stream to the second heat exchanger via the second process inlet;
sending a cooling water stream to the second heat exchanger via second water inlet;
cooling the second portion of the overhead or side stream in the second heat exchanger to yield a cooled second portion of the overhead or side stream, such that the cooled second portion of the overhead or side stream exits the second exchanger second process outlet and the cooling water stream exits the second heat exchanger via the second water outlet;
wherein the temperature of the second process outlet is lower than the temperature of the second process inlet; and
wherein the temperature of the second water outlet is higher than the temperature of the second water inlet.

5. The process of claim 4 wherein the steam generator and the heat exchanger are in series with the first process outlet from the steam generator being sent to the second process inlet of the heat exchanger, and wherein the second portion of the overhead or side stream is the cooled overhead or side stream.

6. The process of claim 4 wherein the steam generator and the heat exchanger are in parallel.

7. A process for the recovery of the heat from a carboxylic anhydride distillation column, comprising the steps of:
providing a crude carboxylic anhydride stream comprising a carboxylic anhydride and a carboxylic acid;
distilling the crude carboxylic anhydride stream in a distillation column having an overhead or side stream comprising purified, vaporous carboxylic anhydride and a bottoms stream;
providing a steam generator comprising a first process inlet, a first process outlet, a first water inlet, and a first steam outlet;
introducing at least a portion of the overhead or side stream to the steam generator via the first process inlet;
introducing a steam condensate stream to the steam generator via the water inlet;
heating the steam condensate stream in the steam generator to yield a low-temperature steam;
cooling the portion of the overhead or side stream at least about 0.1° C. and up to about 190° C. to yield a cooled overhead or side stream;
wherein the low-temperature steam exits the steam generator via the first steam outlet and the cooled overhead or side stream exits the steam generator via first process outlet;
providing a heat exchanger comprising a second process inlet, a second process outlet, a second water inlet, and a second water outlet;
introducing a second portion of the overhead or side stream to the second heat exchanger via the second process inlet;
introducing a cooling water stream to the second heat exchanger via second water inlet;
cooling the second portion of the overhead or side stream at least about 0.1° C. and up to about 190° C. in the second heat exchanger to yield a cooled second portion of the overhead or side stream; and
wherein the cooled second portion of the overhead or side stream exits the second exchanger second process outlet and the cooling water stream exits the second heat exchanger via the second water outlet.

8. The process of claim 7 wherein the first process inlet has a temperature of between 50° C. and 220° C. and the first process outlet has a temperature of between 220° C. and 30° C.

9. The process of claim 7 wherein the first water inlet has a temperature of between 30° C. and 190° C. and the first water outlet has a temperature of between 30° C. and 190° C.

10. The process of claim 7 wherein the steam generator and the heat exchanger are in series with the first process outlet from the steam generator being sent to the second process inlet of the heat exchanger, and wherein the second portion of the overhead or side stream is the cooled overhead or side stream.

11. The process of claim 7 wherein the steam generator and the heat exchanger are in parallel.
